(19) United States
(12) Patent Application Publication
(10) Pub. No.: US 2024/0065675 A1
(43) Pub. Date: Feb. 29, 2024

(54) AUTOMATIC DOPPLER DERIVED BLOOD PRESSURE

(71) Applicant: Gaurang Nandkishor Vaidya, San Mateo, CA (US)

(72) Inventor: Gaurang Nandkishor Vaidya, San Mateo, CA (US)

(21) Appl. No.: 18/380,201

(22) Filed: Oct. 16, 2023

(51) Int. Cl. A61B 8/00; A61B 8/06; A61B 8/08

(52) U.S. Cl. CPC A61B 8/4416; A61B 8/06; A61B 8/4227; A61B 8/463; A61B 8/488; A61B 8/5223; A61B 5/022

(57) ABSTRACT

The present invention comprises a wearable device equipped with sensors capable of obtaining and transmitting ultrasound doppler information. The device includes an integral power source, sensors to send and receive reflected ultrasonic waves, processor to analyze the ultrasonic data and convert to electric signal and one or more human and computer readable output forms. The device is paired with a computing device capable of processing the doppler data and correlating with arterial occlusion pressure to automate the process of measuring doppler derived blood pressure. The obtained pressure readings and heart rate are displayed on a display device. The computing device utilizes artificial intelligence and machine learning of multiple doppler signal iterations to obtain accuracy.

### FIELD OF INVENTION

The present invention is related to the use of a wearable doppler device, and a blood pressure machine endowed with an ability to process the doppler data to measure the blood pressure. More particularly, the invention relates to the use of a wearable doppler technology which can communicate with a blood pressure machine to automate the process of obtaining doppler-derived blood pressure. The invention is intended to provide ambulatory doppler-derived blood pressure, especially in non-pulsatile patients such as those with continuous flow left ventricular assist device.

### BACKGROUND

Assessment of a patient's blood pressure is paramount in making important decisions related to the care of all patients on a regular basis. An accurate estimation is important to make appropriate modifications to lifestyle/medications and follow the effect of treatment. Uncorrected high blood pressure can result in various morbidities including stroke, heart failure, kidney disease etc.

Telehealth has become vital especially since the COVID pandemic and patients have expressed improved satisfaction with telehealth due to the exclusion of travel time to the clinics. The success of telehealth however depends on an ability to mimic clinic-performed evaluations at home. Blood pressure monitoring can now be done at home using commercially available automatic cuffs which rely on oscillometric/vibratory measurements. This technology relies on vibrations detected by the cuff. The cuff initially inflates to a pressure above the systolic blood pressure, effectively compressing all blood flow beyond the cuff. As the cuff deflates gradually to a pressure below the systolic pressure but above the diastolic pressure, the blood flow is reinstated during systole only. This generates an intermittent vibration which is detected by the cuff and noted as the systolic blood pressure. As the cuff deflates further to a pressure below the diastolic pressure, the vibrations disappear. The blood pressure machine notes this as the diastolic blood pressure. The oscillometric technique, thus, relies on the patient having pulsatile flow.

The technique, however, fails in patients who are non-pulsatile, such as patients with continuous flow left ventricular assist device (CF-LVAD). The oscillometric technique also relies on an accurate placement of the cuff on a relatively superficial blood vessel to detect the vibrations; thus, it gives inaccurate readings when an appropriate sized cuff is not used or in patients who may have an anatomically misplaced or deeper-placed artery due to subcutaneous fat. The technique also fails if upper limb arteries are not available such as due to occlusion of the arteries, amputation, vascular disease, arterio-venous fistula, or limb pain.

Doppler derived blood pressure is the gold-standard non-invasive blood pressure monitoring technique in CF-LVAD patients. The current technique involves a manual blood pressure cuff inflated on the limb of the patient. This must be done by another individual who also must hold a doppler probe on an artery distal to the cuff—for example an arm cuff with probe held on the brachial artery. First the individual locates the distal artery using the doppler machine. The machine makes a screeching sound when placed over a blood vessel which corresponds with the underlying blood flow. The individual then manually inflates the cuff using a bulb to a pressure above which the doppler noise halts, indicating an occlusion of distal blood flow. As the cuff pressure falls below the mean blood pressure in the non-pulsatile patient, the doppler speaker generates sounds indicating reinstatement of blood flow. At this time, the individual manually notes the pressure displayed on the manometer gauge as the mean pressure.

Thus, the technique relies on another person taking the pressure manually, while holding the bulb in one hand and the doppler probe in another. The patient cannot take this by themselves as they must hold one arm steady for accurate pressure measurement. This would often require training of a non-medical caregiver to identify the doppler signal/sound, possibly affecting the accuracy. The same issues would also arise if doppler technology is used to measure systolic blood pressure for any of the above-mentioned conditions that make the current oscillometric technique inaccurate.

With emergence of telehealth, an ability to perform remote accurate measurements of blood pressure can be vital for continuity of care. A need exists to automate the doppler-derived blood pressure measurements to improve the accuracy of blood pressure measurements by patients, especially when a caregiver is not available. There would be no learning curve to perform doppler related blood pressure measurements at home, as the artificial intelligence endowed machine would identify and process the doppler signals. Automated measurements can also help relieve nursing staff for other important tasks when patients needing regular doppler derived blood pressure are admitted, allowing seamless documentation of the vitals in the patient's medical record.

It is known that doppler derived blood pressure is the most accurate non-invasive means of blood pressure monitoring. Automating the technique will allow more patients to adopt the technique for ambulatory monitoring.

### BRIEF SUMMARY OF THE EMBODIMENT

The present invention involves a wearable doppler device, positioned over an artery. In addition, a computing device endowed with an ability to recognize and process the doppler derived data (sound or otherwise direct waveform analysis). A set up of a blood pressure measurement machine which can communicate with the above computing device and display the blood pressure corresponding to the analyzed doppler data.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 illustrates a non-limiting example of a wearable ultrasound doppler device.

FIG. 2 illustrates a closer view of a non-limiting example of a wearable ultrasound doppler device.

FIG. 3 illustrates a non-limiting example of a set up to measure the blood pressure using a computing unit (5).

FIG. 4 illustrates an embodiment of the invention set up

### DETAILED DESCRIPTION OF THE DRAWINGS

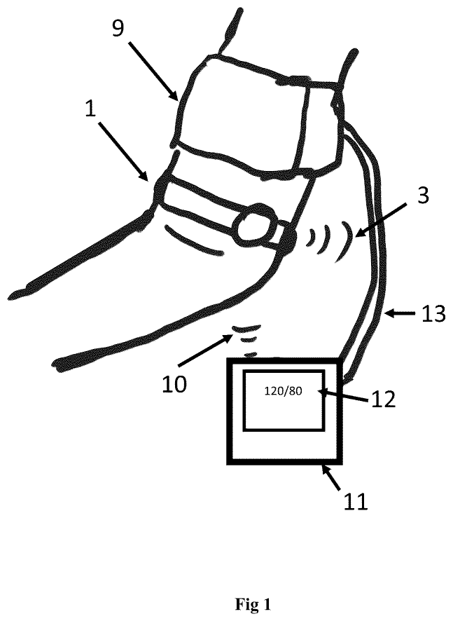

FIG. 1 illustrates a non-limiting example of a wearable ultrasound doppler device (1) which when positioned on the brachial artery (2) would emit sound signals (3) that correspond with the blood flow in the brachial artery. The doppler device is secured around the arm using a strap (4).

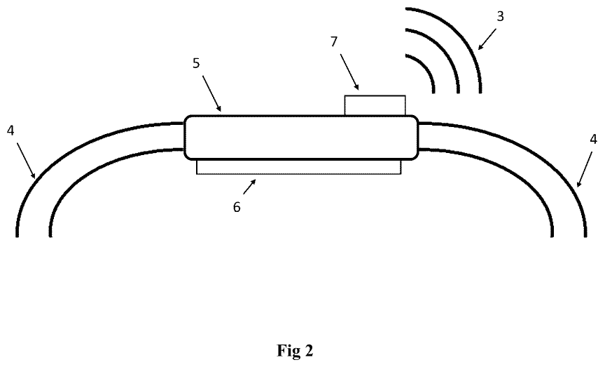

FIG. 2 illustrates a closer view of a non-limiting example of a wearable ultrasound doppler device, consisting of a housing unit (1) with straps to secure on a limb (2), an ultrasonic sensor (3) and a speaker (4) capable of generating sound output (5) generated after processing the doppler signal. The housing unit also contains an integrated power source and a processing unit capable of processing the ultrasonic doppler signal obtained from the sensor (3) into a sound output (5) from the speaker (4). An ultrasound gel is placed by the subject below the sensor (3) to improve its contact with the skin surface.

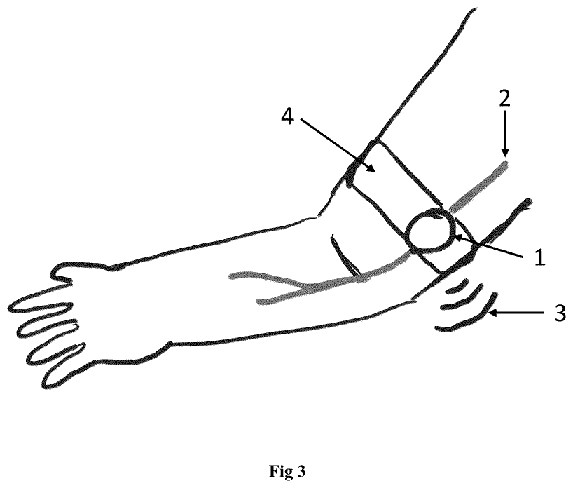

FIG. 3 illustrates a non-limiting example of a set up consisting of a blood pressure cuff (1) positioned proximal to the wearable doppler device (2) on a subject's arm. The doppler device emits sound (3) corresponding to the flow changes in the underlying artery and the sound is received (4) by the computing device (5). The computing device processes the doppler information as noted in the description of the embodiment and displays the blood pressure on the display portal (6). The computing device is connected to the blood pressure cuff through a rubber tubing (7) through which it insufflates air into the cuff and receives cuff pressure related information from the cuff.

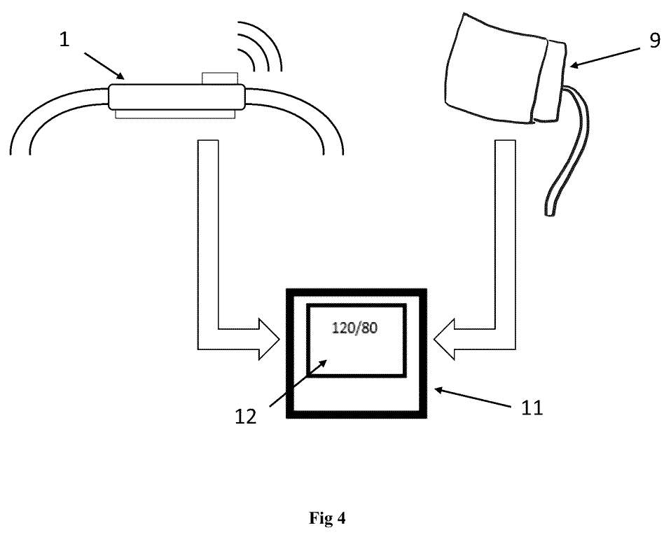

FIG. 4 illustrates an embodiment of the invention wherein the doppler signal received from the wearable device (1) and cuff pressure information from an inflatable cuff (2) are communicated to the computing device (3), which works to correlate the information and display the blood pressure (4).

### DETAILED DESCRIPTION OF THE EMBODIMENT

In the following detailed description, references are made to the accompanying drawings in which are shown the illustrations in how the embodiments may be practiced. It is to be understood that other embodiments may be utilized, with or without structural, procedural or logical changes, without departing from the scope. Therefore, the following description is not to be taken in a restricted or all-inclusive sense, and the scope of embodiments is defined by the appended claims and their equivalents. Moreover, the order of description of various procedures below should not be construed to imply that the procedures are order-dependent.

The description may use perspective-based descriptions such as up/down, proximal/distal, back/front, over/under, anterior/posterior and top/bottom. Such descriptions are merely used to facilitate the discussion and are not intended to restrict the application of disclosed embodiments.

For the purposes of the description, a phrase in the form “A/B” or in the form “A and/or B” means (A), (B), or (A and B). For the purposes of the description, a phrase in the form “at least one of A and B” means (A), (B) or (A and B). The description may use the terms “embodiment” or “embodiments,” which may each refer to one or more of the same or different embodiments.

In the description, various apparatuses are described for use to carry out the various methods within the claims. In the future, a device may be endowed with the doppler and computing ability to perform all or various individual functions together and such a device may be employed to carry out the various methods as described below.

Embodiments described below provide methods to assess a subject's blood pressure using an automated doppler machine. In some embodiment, a wearable device equipped with sensors capable of analyzing underlying blood flow through doppler ultrasound technology is positioned on the brachial artery of a subject (FIG. 1). The device includes an integral power source, sensors to send and receive reflected ultrasonic waves, processor to analyze the ultrasonic data and convert to an electric signal and one or more human and computer readable output forms (FIG. 2). In this embodiment, when the device is positioned over the artery a sound is generated through a speaker within the unit. The sound corresponds to the blood flow in the underlying artery, which is used by the subject to identify the artery. The subject places an ultrasound gel underneath the sensor to improve its contact with the skin surface. In this embodiment, the device is then secured to the limb by the subject using a strap that wraps around the limb and stabilizes the doppler device over the artery. The wearable device makes a sound through a speaker within the unit correlating with the doppler signal.

The further description continues to describe the same embodiment, until noted otherwise. In the same embodiment, the subject secures a blood pressure cuff on the arm proximal to the wearable doppler device (FIG. 3). The cuff is attached to a machine capable of inflating the cuff with air and noting the cuff pressure. This machine contains a computing device which is endowed with an ability to listen and process the doppler sound emitted by the wearable device, through audio recognition. The machine can communicate the cuff pressure with the computing device. In this embodiment, the machine stops inflating the cuff when the cuff pressure reaches 10 mmHg higher than a point when the doppler stops making flow-related sound, indicating occlusion of distal blood flow. This occlusion pressure is identified by the computing device based on its ability to process doppler data.

In the same embodiment, the machine thereafter allows gradual deflation of the cuff pressure by letting the air out from the cuff slowly. Through audio recognition and machine learning, the computing device when the doppler starts making sound intermittently, which correspond with the reinstatement of blood flow in the artery during systole. The cuff pressure at this point is recorded by the device as the systolic blood pressure. The frequency of intermittent doppler sound emission is recorded by the device as the heart rate. As the cuff pressure continues to fall, the doppler sound changes from intermittent to continuous, indicating continuous blood flow reinstatement in the artery. Through audio recognition, the computing device recognizes this continuous doppler sound reinstatement and the corresponding cuff pressure is recorded by the device as the diastolic blood pressure. The recorded blood pressure and heart rate are displayed by the device on a display device.

In non-pulsatile patients, such as those with CF-LVAD, the first reinstatement of blood flow is recorded by the computing device as the mean blood pressure. In such patients, the display device will only display one pressure reading which corresponds to the mean blood pressure.

In another embodiment, the wearable doppler device transmits waveform information to the computing device electronically, either through a wire or through a wireless technology. In this embodiment, the computing device is capable of directly analyzing this doppler waveform. This will be an alternative to the audio recognition.

In another embodiment, all the components (inflatable cuff, doppler and computing devices) are contained within one smart wearable device, which displays the blood pressure on a human readable interface.

The computing device is trained for accuracy by using multiple data points derived from the paired doppler device. The data points consist of doppler signals representing systolic and diastolic blood pressure as noted above, obtained from multiple patients to improve reproducibility.

Given the automatic nature of the device and artificial intelligence involved in recognizing the onset of distal blood flow, the device will allow the patient to take their blood pressure on their own at home, for example.

In another embodiment, the wearable doppler device can also be adopted to locate an underlying blood vessel for applications such as intravenous or intra-arterial access. In some embodiment, the wearable device and/or the computing device are capable of giving the subject feedback on the appropriate positioning over the blood vessel through audio and/or visual means, for example a green light when positioned appropriately over an artery. This is achieved through processing of the doppler signal by the device and the accuracy is improved through machine learning of doppler signals from multiple subjects.

The primary invention thus consists of a novel device equipped with sensors capable of obtaining ultrasound doppler information and transmitting this data in both computer and human readable format. This device is wearable by a subject using a strap or other securing medium. The wearable device frees up the need to hold the doppler probe on the artery by another person.

The invention combines with a novel computing device capable of processing the doppler information and correlating this with arterial occlusion pressure to ascertain the blood pressure independently. This takes away the need for a patient/caregiver to learn doppler interpretation and makes the technique of doppler derived blood pressure measurement less cumbersome and easy to adopt in an ambulatory setting. This method of autonomous doppler derived blood pressure is novel and claimed as an invention here.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein.

What is claimed:

1-30. (canceled)

31. A wearable device, the device comprising:
(a) a housing, wherein the housing encases components of an ultrasound doppler device;
(b) one or more sensors, wherein at least one sensor can acquire ultrasound doppler input;
(c) one or several output forms;
(d) an integral power source;
(e) a processor, and
(f) programming in a non-transitory computer readable medium, wherein the programming is executable by the computer processor and configured to:
(i) receive input from the one or more sensors;
(ii) process the received input into an optimal output form.

32. The device of claim 31, wherein said programming is further configured to:
transmit the optimized output to another computing device through various means including but not limited to audio output generation, direct transmission through wired communication, a wireless technique such as cellular, WiFi, near field communication, infrared or Bluetooth.

33. The device of claim 31, wherein said programming is further configured to:
present the optimized output in a human readable output format such as sound, light or a display device.

34. A computing device, as mentioned in claim 32, the device comprising:
(a) a processor,
(b) a memory,
(c) a display interface,
(d) one or more communications interfaces, and
(e) programming in a non-transitory computer readable medium, wherein the programming is executable by the computer processor and configured to:
(i) receive input from a doppler device;
(ii) receive input from a blood pressure measurement device such as an inflatable cuff.

35. The device of claim 34, wherein said programming is further configured to:
process the received doppler input to identify blood flow within the underlying blood vessel.

36. The device of claim 34, wherein said programming is further configured to:
recognize and register changes in the received doppler signal corresponding to changes in blood flow within the underlying blood vessel.

37. The device of claim 34, wherein said programming is further configured to:
correlate the doppler signal changes with the input received from the blood pressure measurement device to obtain blood pressure and heart rate readings.

38. The device of claim 34, wherein said programming is further configured to:
display the blood pressure and heart rate readings on the display interface in a human readable format.

39. The device of claim 34, wherein the memory is capable of storing, logging and tracking a subject's blood pressure and heart rate over time and displaying this on a display interface device.

40. The device of claim 34, wherein the blood pressure and heart rate data are transmitted to other devices using one or more communications interfaces.

41. A method to evaluate blood pressure and heart rate of a subject, the method comprising: acquisition of ultrasound doppler data of an artery using an overlying wearable doppler device; complete occlusion followed by gradual reinstatement of the blood flow in the artery underlying the wearable doppler device using an external compression device; transmission of the changing doppler signal to a computing device; transmission of the external compression pressure to the same computing device; and the processing of the doppler data by the computing device to obtain the subject's blood pressure and heart rate.

42. The method of claim 41 further comprising utilization of the doppler data to identify an underlying blood vessel.

43. The method of claim 41 wherein, the processing of the doppler data by a computing device comprises utilization of artificial intelligence and machine learning by the computing device to automatically detect changes in the doppler signal during the process of gradual reinstatement of blood flow in the artery and correlating this with the occlusion pressure.

44. The method of claim 41, further comprising displaying the doppler data, blood pressure and heart rate in a human-readable format.
